(19) United States
(12) Patent Application Publication
(10) Pub. No.: US 2020/0398222 A1
(43) Pub. Date: Dec. 24, 2020

(54) CONTROL OF COMPOSITE COVALENT ORGANIC FRAMEWORK BY VARYING FUNCTIONAL GROUPS INSIDE THE PORE

(71) Applicant: UNIVERSITY OF WYOMING, Laramie, WY (US)

(72) Inventors: Katie Dongmei Li-Oakey, Laramie, WY (US); John Hoberg, Laramie, WY (US); Bruce Alan Parkinson, Laramie, WY (US)

(73) Assignee: UNIVERSITY OF WYOMING, Laramie, WY (US)

(21) Appl. No.: 16/901,298

(22) Filed: Jun. 15, 2020

Related U.S. Application Data
(60) Provisional application No. 62/861,626, filed on Jun. 14, 2019.
(60) Provisional application No. 62/933,146, filed on Nov. 8, 2019.

(51) Int. Cl. B01D 61/02; B01D 69/02; B01D 71/72; B01D 71/02; C07F 15/00; C07D 487/22; B01D 53/22

(52) U.S. Cl. CPC B01D 61/027; B01D 69/02; B01D 71/72; B82Y 30/00; C07F 15/0006; C07D 487/22; B01D 53/228; B01D 71/022

(57) ABSTRACT

An ordered functional nanoporous material (OFMN) composition includes a pore defined by a sidewall, the sidewall comprising N—C—N linkages therein. A process for synthesis of a reagent includes the reaction of a 6,7-diaminoquinoxaline having R groups with hexaketocyclohexane (HKH) octahydrate, where R is independently in each occurrence H, Cl, Br, I, C₄H₄S (thiophenyl), SO₃⁻, CO₂⁻, C≡CH, CH═CH₂, NH₂, OH, C≡N, C₁-C₄ alkyl, (CH₂)ₓCH═CH₂, or (CH₂)_{y}CH═CH(CH₂)_{z} where x or (y+z) is an integer of 0 to 4 inclusive, (CH₂)ⱼCH≡CH, or (CH₂)ₖCH≡C(CH₂)ᵣ where j or (k+r) 0 to 4 inclusive. A process of degasification that includes extracting a gas from a mixture by exposing the mixture to an OFNM to selectively pass the gas therethrough. A process of dehydrogenation includes exposing an aliphatically unsaturated feedstock to platinum modified OFNM under conditions to form hydrogen and selectively passing the hydrogen through the platinum modified OFNM.

This application claims priority benefit of U.S. Provisional Application Ser. No. 62/861,626 filed on Jun. 14, 2019 and U.S. Provisional Application Ser. No. 62/933,146 filed on Nov. 8, 2019, the contents of which are hereby incorporated by reference.

### FIELD OF THE INVENTION

The present invention in general relates to filtration membranes and in particular to nanoporous polymeric material membranes with high filtration selectivity and paradoxically high permeance. The present invention additionally relates to materials and the synthesis of new families of Ordered Functional Nanoporous Materials (OFNMs); and in particular synthetic precursors and methods that yield to an entirely new class of ordered two-dimensional (2D) and three-dimensional (3D) nanoporous OFNMs with a unique combination of electronic conductivity, gas transport ability, supercapacitors, catalyst binding, and ion transport properties

### BACKGROUND OF THE INVENTION

The past decade has seen an explosion of interest in two-dimensional (2D) materials that started with the demonstration of the extraordinary properties of graphene, and has been extended to other 2D materials, such as transition metal dichalcogenides, nanoplatelets and other elemental 2D phases (germanene, silicene, etc.).(1) The promise of 2D materials is largely based on their unique single-layer electrical, optical, and magnetic properties. However, current 2D materials are not easily modified to suit a given application: that is, there is very little flexibility in adjusting the materials performance beyond their intrinsic properties. This rigidity and lack of adaptability presents significant barriers to technological implementation and broad use. Attempts have been made to achieve this goal by modifying graphene. For example, a top down approach using ion bombardment(2), etching(3), or oxidations(4), produces graphene oxide (GO) with pores containing a high degree of polydispersity in both size and density. These randomly produced pores start to overlap when produced at high density producing both larger openings and weakening the material. In fact, variations in the degree of oxidation caused by differences in starting materials (principally the graphite source) or oxidation protocol can cause substantial variation in the structure and properties of the material.(5) As a result, permeation (flux) through GO membranes remains insufficient to technically compete with current commercial pressure-driven membranes.(6) This challenging task of creating atomically precise nanopores, without destroying the material itself, has thus remained elusive. However, just recently a bottom-up synthesis of a nanoporous “graphene” was reported,(7) providing a material with ordered nanopores while maintaining the integrity of the graphene. Although this bottom-up strategy proved to be successful in the monolayer regime, the nine-step synthesis provides only nanogram quantities and did not produce a material capable of pore functionalization. Metal organic framework materials have also been investigated for membrane production however they suffer from their 3D structures where membranes have to be fabricated with grains of these materials where species can diffuse in the spaces between grains rather than through the porous structure. A 2D material can naturally produce a membrane without this possibility via the natural stacking of the 2D grains as in graphene oxide where the size selection has actually been attributed to the tortuous diffusion path between the layers. These ordered and completely engineered pores might have great efficacy across multiple applications, including high performance separations.

Separations are fundamental to life processes, analytical protocols, industrial processes and consumes greater than 10% of world energy use. (10) Many of the conventional separation techniques, such as distillation, extraction and chromatographies, are both time and energy intensive. In addition, ion or gas permeable membranes are vital to the operation of virtually all electrochemical devices including batteries, fuel cells, electrolyzers and desalinization systems. Additionally, it is well known in the art that the relationship of throughput and selectivity of a filter is generally inversely proportional.

The demonstration that single layers of graphite or graphene have extraordinary properties including specific optical activity, high carrier mobility, and high electrical and thermal conductivity, makes graphene a very promising candidate material for nanoelectronics. Graphene is a lattice of carbon atoms so thin that graphene is considered to be two-dimensional (2D) having sp²-hydridized carbon atoms that afford planar conductivity. However, graphene is missing the typical electronic band gap that would make it a semiconductor. The electronic band structure of graphene resembles relativistic particle-antiparticle pairs. Graphene as a prototypical two dimensional (2D) material has initiated an explosion of interest in 2D materials, including the layered structure transition metal dichalcogenides with the prototypical MoS₂ structure, which are changed and sometimes enhanced properties at the single layer level. Substitutions of heteroatoms into the graphite structure can produce other 2D materials such as the isoelectronic, yet insulating, boron nitride that has improved strength and thermal characteristics relative to graphene. Simple substitution of nitrogen for carbon in graphene-like structures has resulted in several other ordered 2D OFNMs such as g-C₃N₄, C₄N, C₄N₃ and the much more recent ‘C₂N holey 2D crystal’ or ‘C₂N-h2D’; however, most reports of nitrogen containing graphitic materials have no long range order or controllable pore size.

So-called porous covalent triazine-based framework (CTF) materials (C₃N) have been synthesized by polymerization of aromatic dinitriles in a ZnCl₂ molten salt at temperatures between 300 and 600° C. The prototypical CTF material used p-dicyanobenzene as a reagent to synthesize CTF1 is shown in prior art FIG. 1, and several other dinitriles have been employed to produce variations on this structure. Furthermore, various CTF frameworks have been made from p-dicyanopyrimidine and other aromatic ring systems such as 1,5 dicyanopyridine to form a CTF with the stoichiometry of C₂N₃ (prior art FIG. 2). These materials were mostly disordered, but calculations of the electronic properties of the ideal framework showed that these materials would be semiconducting.

There has also been considerable work on the C₃N₄ systems synthesized from a variety of routes, but most frequently from melamine polymerization. Most of these syntheses produced disordered materials until Algara-Siller et al. in 2014 made the first highly ordered material that could then be designated as g-C₃N₄, where g is for graphitic. (G. Algara-Siller et al., Angew. Chem. 2014, 126, 7580-7585). In the synthesis described by Aglara-Siller et al., a molten salt was used with dicyandiamide heated to 600° C. for 60 hours in a LiBr/KBr eutectic sealed in a quartz ampule. Two different allotropes of g-C₃N₄, the triazine and heptazine based structures were produced from this reaction as seen in prior art FIG. 3. The triazine and heptazine based structures have semiconducting properties and have been examined for potential photovoltaic and photocatalytic properties.

The new g-C₂N materials are of particular interest since they are made using a simple well-known condensation reaction between a ketone and amine at lower temperatures to form imine moieties, specifically hexaminobenzene and hexaketocyclohexane (HKC) in a highly exothermic reaction (−89.7 kcal/mole from DFT) to form the g-C₂N, as shown in prior art FIG. 4. This material is semiconducting with a direct band gap of ˜1.7 eV. It was also shown that these 2D crystals have an optical gap of 1.96 eV. The DFT calculations that have been performed revealed flat bands near the edges of the conduction and valence bands indicating a high level of delocalization of the π-states and implying high mobilities that were also measured in single layer FET devices. Interestingly, the valence band is doubly degenerate at the Γ-point. Such degeneracy can be removed by a pseudo-Jahn-Teller effect where electrons interact with phonons resulting in the buckling distortions of the 2D sheets. Annealed samples were also shown to be highly ordered and in a graphitic layered structure and were stable in argon to 900° C. and in air to 550° C. Field effect transistors (FETs) made from single flakes of g-C₂N had exceptional properties with an average maximum on/off ratio between the maximum and minimum drain currents obtained from 50 devices of 4.6×10⁷. The g-C₂N material also has high electron and hole mobilities of 13.5 cm²/V-s and 20.6 cm²/V-s respectively on what is likely a rather defective material.

Recently, Guo et al. have reported a conjugated organic framework with a delocalized pi-electronic structure, yet one that lacks additional reactive moieties. Guo, J. et al.; Conjugated organic framework with three-dimensionally ordered stable structure and delocalized π clouds, Nature Communications 4, Article number: 2736 (2013) doi:10.1038/ncomms3736.

Considerable progress has been made in producing OFNMs that are readily synthesized with a controlled pore sizes and pores that can be chemically modified as detailed in PCT/US17/20000. Such materials have proven to be highly effective as separation membranes as detailed in PCT/US19/46528.

However, needs still exist as to pores containing N—C—N bonds defining a pore side wall. There further exists a variety of applications that are poorly served by current materials for which OFNMs are well suited.

Accordingly, there exists a need for a membrane for separations that has both high throughput and highly selective transport or rejection of the species of interest based on size, charge or other molecular properties.

### SUMMARY OF THE INVENTION

The present invention provides an ordered functional nanoporous material (OFMN) composition comprising a pore defined by a sidewall, the sidewall comprising N—C—N linkages therein. A process for synthesis of a reagent is also provided that includes the reaction of a 6,7-diaminoquinoxaline having R groups with hexaketocyclohexane (HKH) octahydrate, where R is independently in each occurrence H, Cl, Br, I, C₄H₄S (thiophenyl), SO₃⁻, CO₂⁻, C≡CH, CH═CH₂, NH₂, OH, C≡N, C₁-C₄ alkyl, (CH₂)ₓCH═CH₂, or (CH₂)_{y}CH═CH(CH₂)_{z} where x or (y+z) is an integer of 0 to 4 inclusive, (CH₂)ⱼCH≡CH, or (CH₂)ₖCH≡C(CH₂)ᵣ where j or (k+r) is an integer of 0 to 4 inclusive. Additionally, provided is a process of degasification that includes extracting a gas from a mixture by exposing the mixture to an OFNM to selectively pass the gas therethrough. Also, a process of dehydrogenation is provided that includes exposing an aliphatically unsaturated feedstock to platinum modified OFNM under conditions to form hydrogen and selectively passing the hydrogen through the platinum modified OFNM.

### BRIEF DESCRIPTION OF THE DRAWINGS

The application file contains at least one drawing executed in color. Copies of this patent application publication with color drawings will be provided by the Office upon request and payment of the necessary fee.

The present invention is further detailed with respect to the following drawings. These figures are not intended to limit the scope of the present invention but rather illustrate certain attributes thereof.

FIG. 1 shows a schematic representation of COF structures and the fabrication of layer-by-layer (LbL)-COF/anodic aluminum oxide (AAO) composite membrane in accordance with embodiments of the invention;

FIGS. 2A-2C illustrate charge manipulation of a zwitterion containing pore where FIG. 2A is a zwitterion form, FIG. 2B is a positively charged pore, and FIG. 2C is a negatively charged pore in accordance with embodiments of the invention;

FIGS. 3A and 3B illustrates an example of a second generation zwitterion with reduced pore size and tuning of the zwitterion window in accordance with an embodiment of the invention;

FIGS. 4A-4C illustrate the cross-linking of a first COF structure (COF 1) and a second COF structure (COF 2) in accordance with embodiments of the invention;

FIGS. 5A and 5B illustrate a schematic representation of the application of a scanning electrochemical cell microscope for the investigation of thin single particle 2D-COF membranes in accordance with embodiments of the invention;

FIG. 6 is a prior art synthesis process for single pore COFs that uses pyrene tetraone;

FIG. 7 illustrates a reaction for yielding diester pyrenetetraone (DEPTO) in accordance with an embodiment of the invention;

FIG. 8 illustrates a prior art reaction for the synthesis of the Biphenyl adduct;

FIG. 9 illustrates a prior art reaction that couples 2,6-diformic acid Me ester-1-bromobenzene with copper powder in organic solvent DMF to yield pyrene tetraone;

FIG. 10 illustrates a prior art procedure for the bromination of pyromellitic acid;

FIG. 11 illustrates a reaction for formation of TAPS;

FIG. 12 illustrates the reacting of TAP with HKH to produce two isomers for use in COF formation;

FIG. 13 illustrates an amino alcohol based COF in accordance with embodiments of the invention;

FIG. 14 illustrates a prior art synthesis of reagent a 6,7-diaminoquinoxaline (DAQX);

FIG. 15 illustrates an inventive synthesis reaction of DAQX with hexaketocyclohexane (HKH) octahydrate to form a triquinoxaline precursor for an inventive OFNM;

FIG. 16 illustrates an inventive synthesis of a novel OFNM from a triquinoxaline and a hexaazatriphenylenehexacarbonitrile (HAT-hexacarbonitrile), the box highlights the N—C—N linkage in the pore sidewall;

FIG. 17 illustrates a prior art reaction to form (HAT-hexacarbonitrile);

FIG. 18 illustrates the reaction scheme highlighting the variable moieties, R projecting into the pore volume and external to the pore;

FIG. 19 illustrates an inventive synthesis of a novel 2 pore type OFNM based on a Heck coupling of a triquinoxaline precursor as reagent for a novel OFNM synthesis with functionalized pores;

FIG. 20 illustrates the reaction scheme a synthesis of a functionalized OFNM through a reaction scheme involving a functionalized tetraminobenzene and a triquinoxaline precursor;

FIG. 21 illustrates a reaction scheme for a novel dual pore OFNM;

FIG. 22 illustrates a reaction scheme to insert a metal ion into the dual pore OFNM of FIG. 21; and

FIG. 23 is schematic showing dehydrogenation and separation of hydrogen gas using an OFNM.

### DESCRIPTION OF THE INVENTION

The present invention has utility in providing a method to synthesize new families of Ordered Functional Nanoporous Materials (OFNMs) that can be manipulated with targeted organic synthesis. As used herein, OFNMs are synonymously referred to herein as two-dimension OFNMs or nitrogen containing graphitic materials (NCGMs). The OFNMs so produced represent a range of new functional materials applicable to: selective ion transport membranes, selective gas transport membranes, battery electrodes, electrolyzer electrodes, fuel cell electrodes, desalinization systems, bipolar membranes, field-effect transistors, sensors, filters, supercapacitors and chemical and electrochemical catalysis. An inventive reaction scheme provides for self-assembly of inventive materials with long-range order.

The present invention further provides filtration membranes with high filtration selectivity based on specific chemical properties such as size and charge while also affording high permeance. The membranes of the present disclosure are attractive separators due to their small energy requirements and their potential for both fast and selective separations. Membranes according to embodiments of the present disclosure have atomic scale capillaries that efficiently allow the separation of the species from solutions and suspensions based on properties depending on the molecular and ionic size.(11) According to some inventive embodiments, a membrane is fabricated from A covalent organic framework (COF). As a result, solvent permeance values of more than 900 Lm⁻²h⁻¹bar⁻¹ are achieved and in some inventive embodiments, values of between 900 and 6000 Lm⁻²h⁻¹bar⁻¹ are achieved. In concert with the permanence values obtained through use of an inventive filter, filtered species rejection percentages are achieved that are greater than 60% and in some inventive embodiments between 60 and 95% per single membrane pass.

The present invention provides a novel class of two-dimensional covalent organic framework (COF) polymers that have a highly stable, photoactive, semi-conducting aromatic backbone with intrinsically and exactly ordered nanometer sized pores, and, unlike other COFs,(8, 9), can be functionalized with a variety of functional groups. According to some inventive embodiments, a highly ordered COF is synthesized with ionizable carboxylate groups in 2.8 nm pores and demonstrates high membrane selectivity to only conduct cations smaller than a precise pore size threshold. Additionally, related inventive membranes materials are readily synthesized to either increase or reduce this pore size threshold or make yield anionic selective membranes. These 2D-COF materials achieve the goal of a modifiable, highly ordered material and are synthesized in a bottom up approach, thereby providing both a stable aromatic backbone and producing functionalized pores either in the small precursor molecules or after synthesizing the COF using well known high yield coupling reactions to replace moieties extending into pore areas with substituted moieties so as to modify pore properties. Substituted moieities operative herein illustratively include halogens, amines, hydroxyls, carboxyls, peptides, ammoniums, oniums, alkanes, alkenes, silanes, sulfonyls, and phosphates. It is appreciated that with resort to chiral substituted moieties that chiral selectively is imparted to an inventive membrane.

It is also appreciated the pore moieties are also selective reacted with a cap species, to selectively close a pore. In instances when the cap species is a precious metal or contaminating metal present in low concentrations such as radioactive contaminants, an inventive membrane serves as a cap species accumulator.

The term “nanopore” is used herein synonymously with pore and intended to define a central void with a longest linear dimension in the plane of an inventive OFNM ranging from 1.2 nanometers (nm) to upwards of 82 nm.

As used herein, an aromatic, a heteroaromatic, an amino acid, a glycol, a sugar, are defined as each having a molecular weight of less than or equal 300 atomic mass units.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

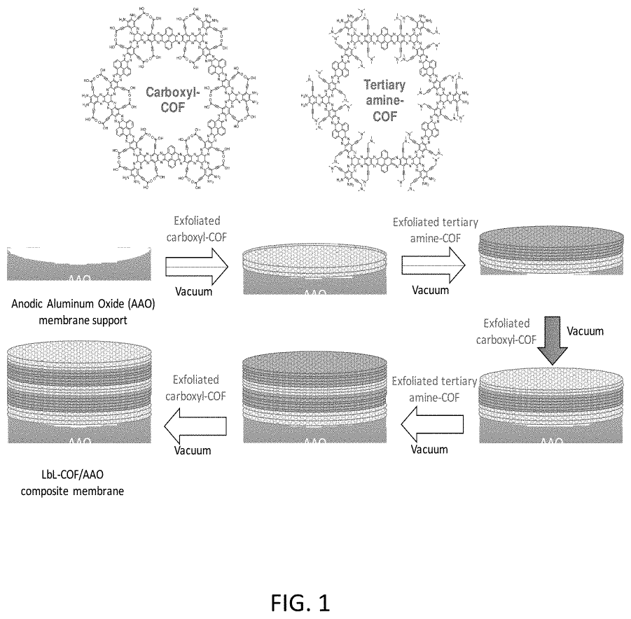

Embodiments of the invention provide control of composite covalent organic frameworks (COF) by varying functional groups inside the pore of the COF. In a specific inventive embodiment a COF membrane consisting of both a carboxylated COF (C-COF, hydrophilic) and tertiary amine lined pore (N-COF, hydrophobic) supported on an anodic aluminum oxide (AAO) substrate with an alternative layer-by-layer (LbL) was constructed as shown in FIG. 1. In FIG. 1 an anodic aluminum oxide (AAO) substrate is a membrane support on which an exfoliated carboxyl COF is deposited under vacuum conditions. In subsequent steps exfoliated tertiary amine COF and exfoliated carboxyl COF are applied in alternating layers as a stack on the AAO substrate that forms a LbL-COF/AAO composite membrane. It has been determined that the composite LbL COF membranes disintegrate in water, while the composite LbL COF membranes are stable in organic media, including methanol, N,N-dimethylformamide (DMF), and ethanol

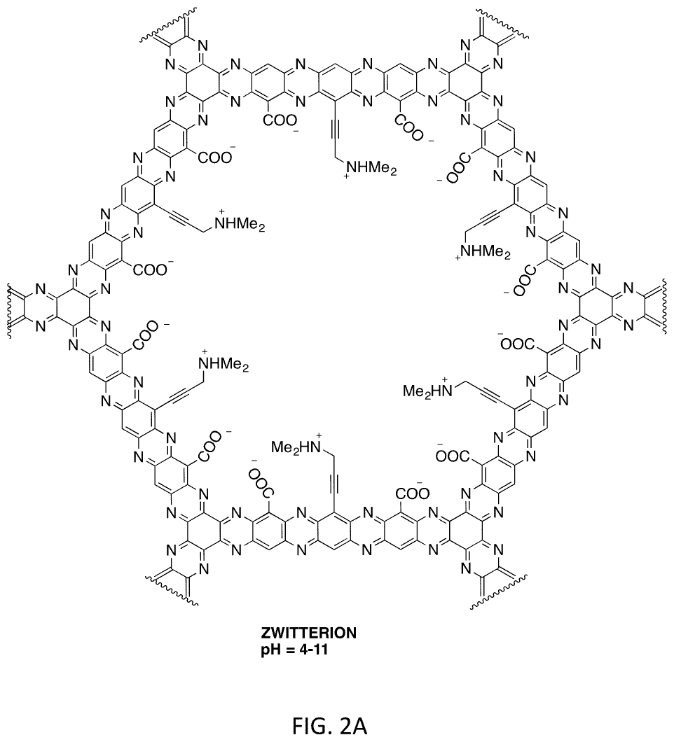

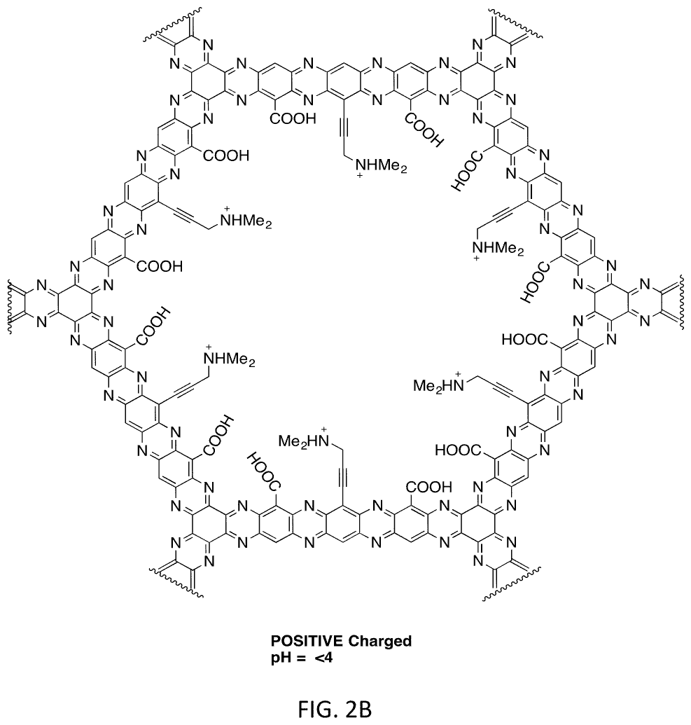

In specific inventive embodiments multilayer COF membranes may be formed using the mixed zwitterion 1E with the single pore by a simple combination of carboxylated groups and amines. A zwitterion is a molecule with two or more functional groups, of which at least one group has a positive and one group has a negative electrical charge and the net charge of the entire molecule is zero. As shown in FIGS. 2A-2C the pH levels determine the charge of the molecules. In FIG. 2A at pHs between approximately 4-11, the zwitterion form will exist and provide strong hydration through electrostatic interactions with water molecules, while simultaneously providing a physical and energetic barrier against, for example, protein adsorption. The secondary structure assumed by proteins produces a heterogeneous but characteristic distribution of surface charges that largely dictates their ability to bind to surfaces. Generally, proteins can only bind to surfaces with a uniform charge. Surfaces that display heterogeneous charge density, for instance, zwitterionic surfaces, demand that proteins modify their structure (denature) to conform to the surface charge density in order to adsorb. Consequently, proteins are thus prevented from binding or, in some cases, repelled from the surface with heterogeneous charge density. Alternatively, adjusting the pH to less than 4 (<4) will produce a positively charged pore as shown in FIG. 2B, allowing passage of anionic substrates. Finally, a pH greater than 11 (>11) will produce a negatively charged pore (FIG. 2C) to allow passage of cations. In inventive embodiment the ranges may be fine-tuned by using inductive effects on both the amine and carboxyl moieties. For example, replacement of the N,N-dimethylpropynylamine with para-ethynyl-N,N-dimethylaniline will not only change the pkₐ of the protonated amine from a pH of approximately 11 to approximately 5.5, but will greatly reduce the pore size to 1.2 nm as shown in FIGS. 3A and 3B. FIGS. 3A and 3B show an inventive example of a second generation zwitterion with reduced pore size and tuning of the zwitterion window. Furthermore, switching the COOH with CH═CHCOOH moves the acid pkₐ from approximately 4 to 2.

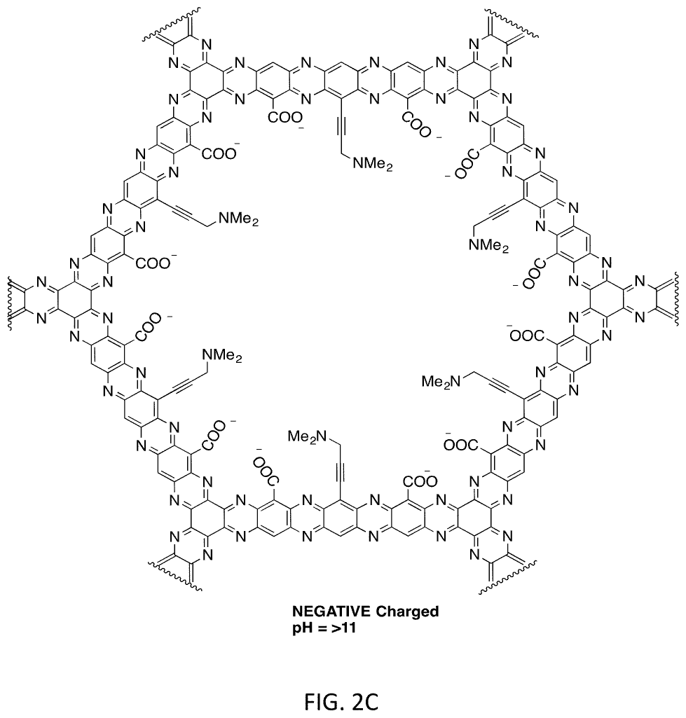

In an inventive embodiment, an additional modification that allows further solid-liquid interactions is to cross-link the two dimensional (2D) COF sheets. FIGS. 4A-4C illustrate the cross-linking of a first COF structure (COF 1) and a second COF structure (COF 2). In FIG. 4A, the sheets of COF 2 are cross-linked via metal-ligand binding. This cross-linking aligns the channels as illustrated with the wavy lines. Secondly in FIG. 4B, cross-linking of COF 1 is achieved via esterification of carboxyl groups. Using a mixed COF that incorporates a carboxylic acid moiety in the pore, an acid-catalyzed esterification using reagents, illustratively including ethylene glycol, imparts connectivity between layers as shown in FIG. 4C. In both examples, only a small level of cross-linking needs to be accomplished (approximately 5%) to form the desired channels.

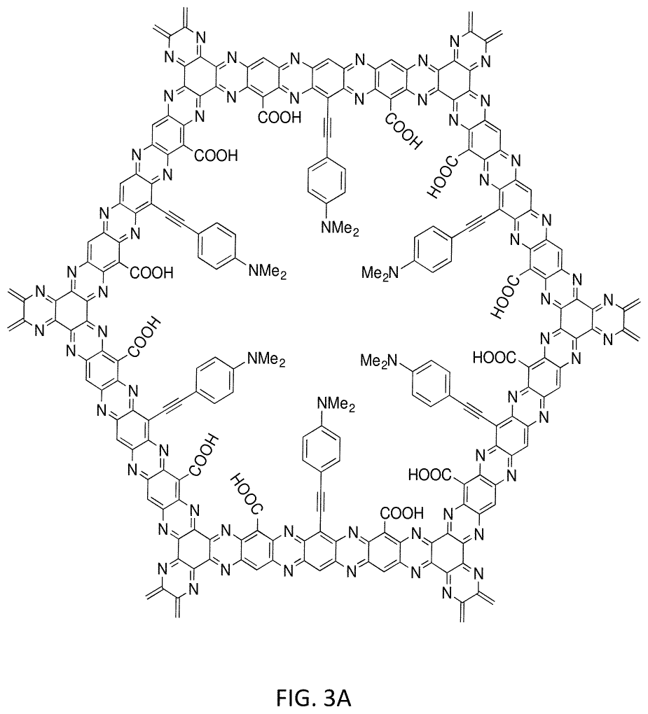

Embodiments of the inventive COF-based membranes may have both high selectivity and permeability for a few different liquid separations. The fundamental limits of these parameters have smaller and thinner membranes with the goal of measuring properties of the ultimate single layer crystalline flake such that have dimensions on the order of several hundred nanometers on a side. In specific inventive embodiments, using seeding techniques the size of single crystal sheets has been increased by introducing highly ordered small sheets into the reaction mixture to favor grain growth rather than new nucleation to produce flakes up to many microns in diameter. Ion transport measurements using single layer membranes were made to resolve the controversy of whether graphene itself was a proton specific membrane due to tunneling through the middle of the benzene-like rings in graphene. The experimental details are given by Hu et al in the supplementary information of their recently published work. (citation needed) Briefly, this reported technique mounted micrometer scale sheets of graphene onto pulled micropipettes immersed in an electrolyte to establish that defect free layers did not conduct protons and that when small ion currents were measured, the small ion currents could be associated with defects in the graphene layers. In inventive embodiments, a similar technique is used to measure the fundamental maximum of ion conductivity and selectivity of the inventive COF membranes using small crystalline sheets of COF materials. Specifically, a pipette puller was used to produce the micro- and nano-meter pore sizes in glass capillaries as was done in references (49), (50). However, unlike that experiment, where the hydrophobic graphene was floated on top of the electrolyte, the hydrophilic membrane flakes are supported on cylindrical Vycor glass with approximately 1 millimeter thickness as shown in FIGS. 5A and 5B, since the COF membranes may not float on electrolyte solutions, due to their hydrophilic nature. Vycor is a nanoporous glass with 1 to 10 nm pores making up 25-30% of the glass volume that has been used as an inert ion conducting media for isolating reference electrodes in electrochemical cells. Unlike the previously reported experiments, real-time imaging capabilities were used to measure the magnitude and uniformity or non-uniformity of the ion currents over the surface of the flakes using scanning electrochemical cell microscope (SECCM). A diffraction limited optical microscope is integrated into the SECCM that enables the identification of single and multiple flake regions of the single crystal COF flakes deposited on the substrate in a similar manner used to prepare TEM grids for COF imaging. By changing the composition of the electrolytes in the pipette and the reservoir, an ability to change the size and concentrations of both anions and cations to make groundbreaking measurements was achieved, which allowed for the investigation of the fundamental limits of both flux and selectivity for various ions in these novel COF membrane materials.

FIGS. 5A and 5B illustrate a schematic representation 10 of the scanning electrochemical cell microscope used in the investigation of thin single particle 2D-COF membranes. In FIG. 5A a pulled micro or nano sized pipette tip 12 is filled with an appropriate electrolyte solution 14 that is rastered over single 2D-COF flakes 16 supported on a polished ion conducting nanoporous Vycor glass surface 18. The change in the ion current is then measured as a function of position as compared with the higher ion flow when the tip is over a bare Vycor region. FIG. 5B is an expanded view of the tip region 12 showing the small electrochemical cell volume where ion flow can be measured as a function of position to determine the influence of the number of 2D-COF layers on the ion current. Judicious choice of electrolytes 14 in the Vycor and the pulled pipette 12 demonstrate the ultimate selectivity of the membrane for various sizes and charges of ions as a function of membrane structure and order.

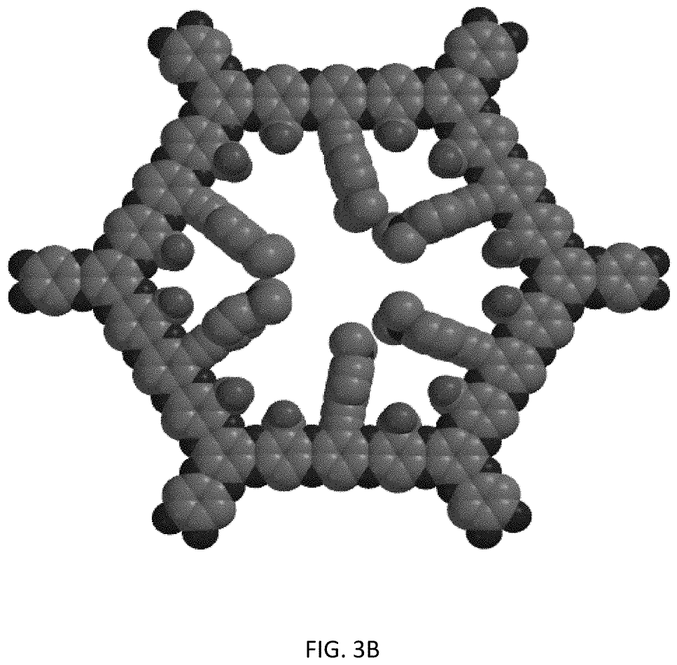

Embodiments of the invention provide a new alternative pyrene tetraone synthesis. For the current construction of the single pore COFs, pyrene tetraone is used. Pyrene tetraone is synthesized in approximately 10-15% yields (FIG. 6) using a published procedure (14) which is then derivatized in multiple steps to put R groups on (e.g. Br, COOH).

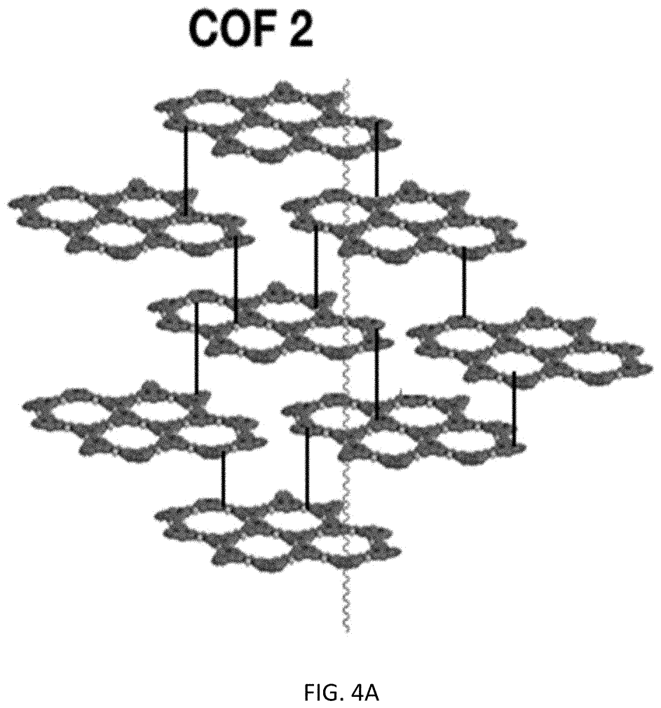

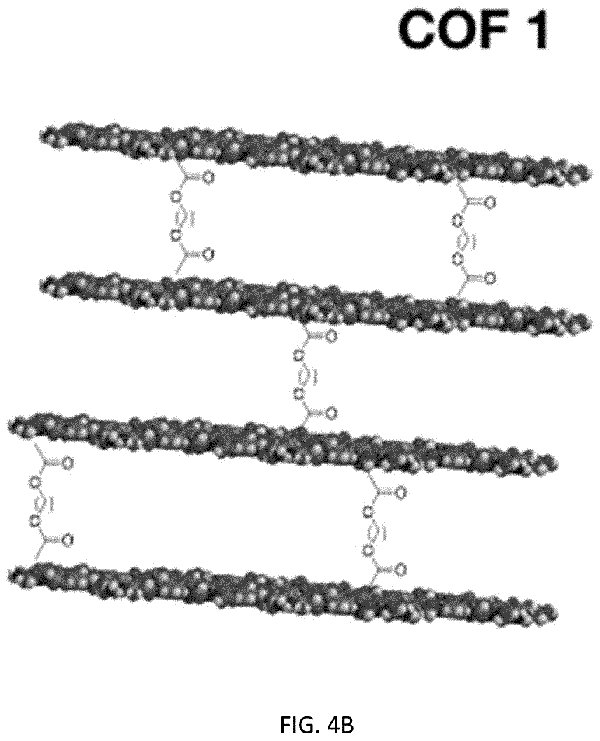

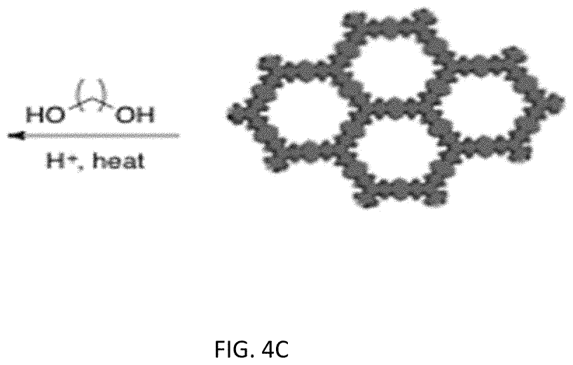

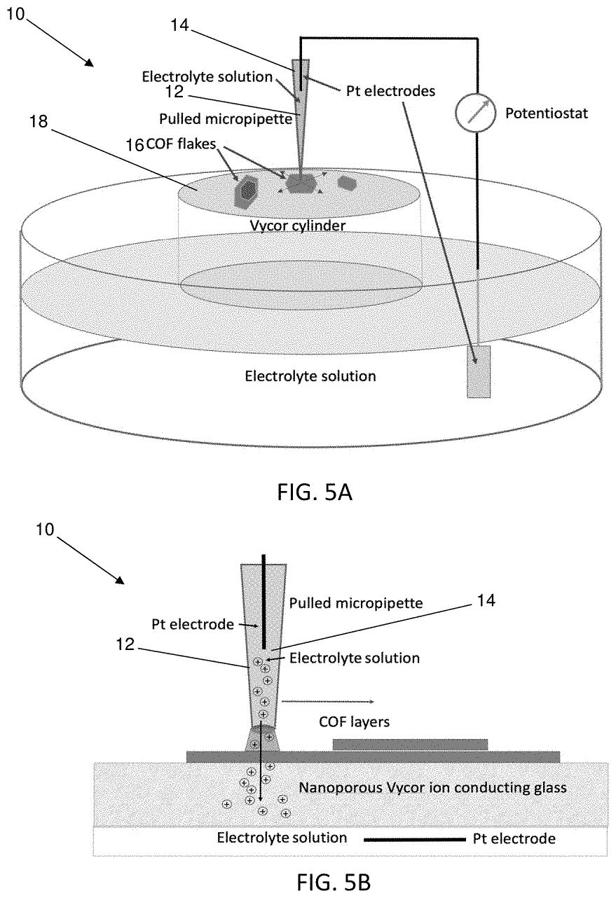

The new target is DEPTO (diester pyrenetetraone) and the overall synthesis of DEPTO is outlined in FIG. 7, which includes two new routes to the Bromo Trimesic ester. Mesitylene derivatives illustratively including Bromomesitylene and Trimesic acid are commercially available and are very inexpensive. The reaction shown in FIG. 7 is based on two published procedures. The first procedure is outlined in FIG. 8 for the synthesis of the Biphenyl adduct. (15) The KMnO4 is in seven fold excess and generates large amounts of waste by-products and thus is very undesirable for scale-up. The yield is also rather poor. Thus, in inventive embodiments only the Cu step is used. The second procedure is shown in FIG. 9 and is based on a Chinese patent (CN102617317A). The bis(salicylidene)ethylenediamine cobalt is used as a catalyst (second step). The starting material in the second procedure is expensive and thus the use of the above mesitylene route is a very inexpensive alternative. Finally, the proposed bromination of trimesic acid is based on a procedure for the bromination of pyromellitic acid shown in FIG. 10. (16)

According to embodiment, 0.64 g (0.0025 mol) of pyromellitic acid and 1.60 g (0.01 mol) of bromine was heated in mixture of 12 ml of concentrated sulfuric acid and 5 ml of nitric acid at 75° C. for 15 h. The solution was poured in ice water (100 g), precipitate was filtered off, washed with water and dried. 3-bromopyromellitic acid was obtained in yield 0.75 g (90%).

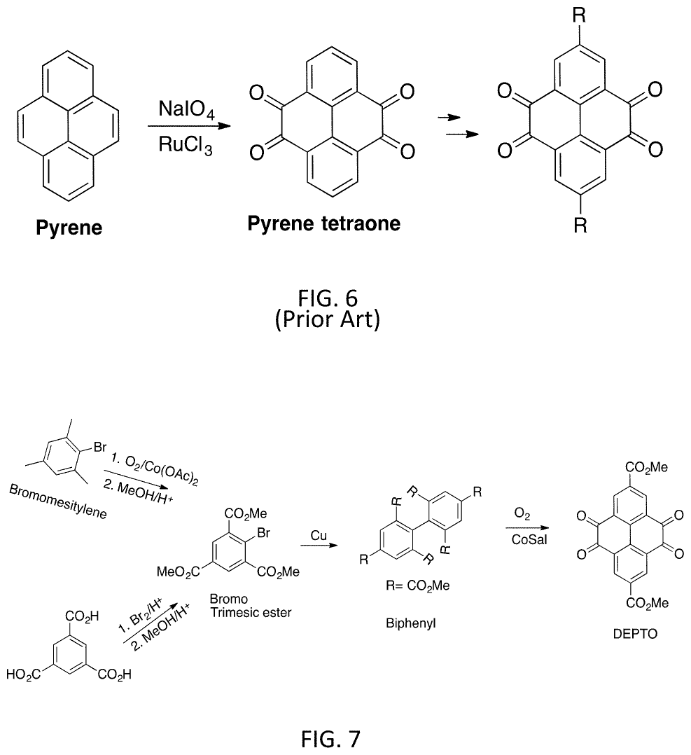

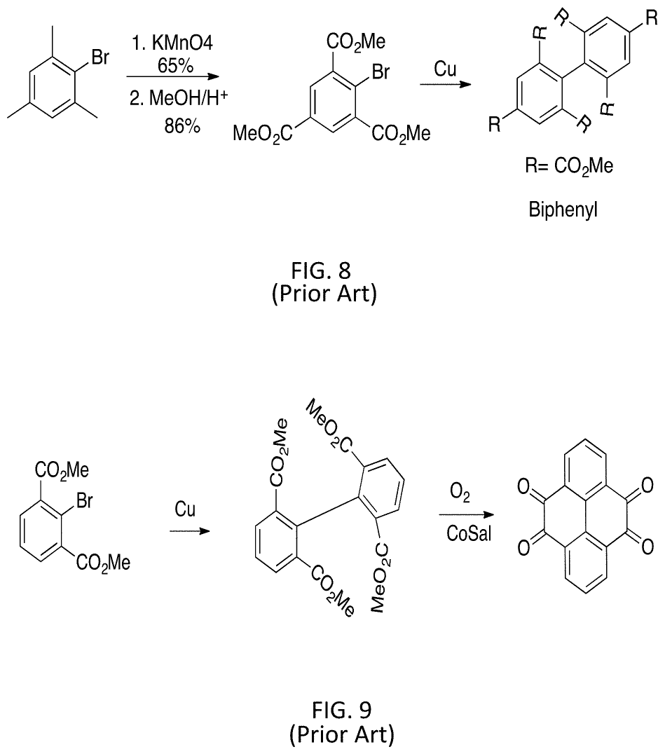

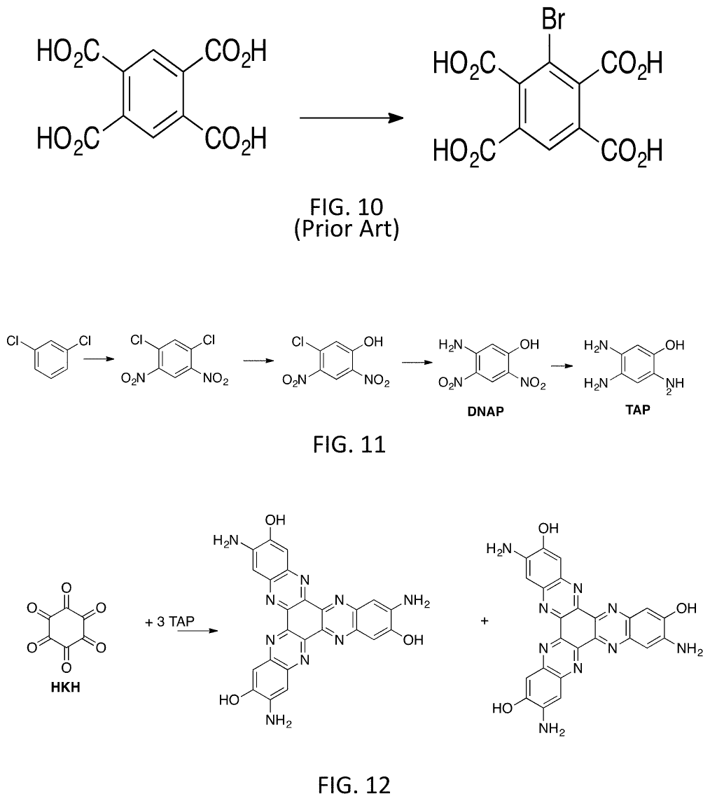

An inventive amino alcohol based COF is also provided as shown in FIG. 13. FIG. 11 illustrates a reaction for formation of TAPS. TAPS is a chemical compound commonly used to make buffer solutions. TAPS can bind divalent cations, including Co(II) and Ni(II). TAPS is effective to make buffer solutions in the pH range 7.7-9.1, since it has a pKₐ value of 8.44. FIG. 12 illustrates the reacting of TAP with HKH to produce two isomers for use in COF formation. As shown in FIG. 13 the COF forming reaction is based on the two isomers reacting with 2,5-dihydroxy-1,4-quinone.

In a specific inventive embodiment a positively charged Pd precursor, illustratively including Pd aquo 2+, is infused, and is bound by the carboxylates and is then reduced to Pd nanoparticles that will be stabilized by the multiple carboxylate groups in the pores of the COF and acts to prevent ripening of very small particles but still allowing rapid ingress and egress of hydrogen into the bulk of the material. This is a huge advantage over bulk Pd hydride but also may be an advantage for hydrogen separation. This is of value for hydrogen storage.

All filtration tests are performed at room temperature under a trans-membrane pressure of 1 bar, using a dead-end permeation cell with an effective membrane diameter of 1 cm.

Solvent permeance (Lm⁻²h⁻¹bar⁻¹) and filtered species rejection (%) values are measured to evaluate the membrane separation performance. A solvent operative herein illustratively includes, water, any organic solvents compatible with a given membrane support, gases, and super critical carbon dioxide. It should be appreciated that the COF from which the layer is formed are exceptional stable under a variety of solvents and at elevated temperatures. Filtered species according to the present invention are also a broad class that includes molecules; ions; macromolecules, such as polypetides, proteins, viruses, bacteria, nanocrystals, colloids, and combinations thereof with the proviso of being sized and/or charged relative to the pores of the two dimensional layer. By way of example, water permeance is calculated by Equation 1.

$$Water permeance = \frac{Δ V}{Δ t {A}_{e f f} Δ P} \qquad Equation 1$$

where ΔV (L) is the volume of deionized water that has permeated through the membrane in a predetermined time Δt (h), A_{eff} is the effective membrane surface area (m²), ΔP is the trans-membrane pressure (bar).

Membrane selectivity is illustratively evaluated for a filterable species being the protein separation ability of membranes using 1000 ppm bovine serum albumen (BSA) protein in phosphate-buffered saline (PBS) solution as a feed. The protein rejection (%) is calculated by Equation 2.

$$Rejection = ( 1 - \frac{{C}_{p}}{{C}_{r}} ) \times 100 \% \qquad Equation 2$$

where Cₚ and Cᵣ are the BSA concentration in the permeate and retentate, respectively. BSA concentration is determined by a SpectraMax Plus 384 UV-Vis (Molecular Devices) from the absorption value at 280 nm.

Neutral solute separation is used to determine the pore size distribution, mean effective pore size (μₚ), and molecular weight cut-off (MWCO) of membranes. An aqueous solution containing PEG (Mw=10,000 g mol⁻¹ and Mw=35,000 g mol⁻¹) and PEO (Mw=100,000 g mol⁻¹ and Mw=400,000 g mol⁻¹) at a concentration of 50 ppm each solute. The solute rejection is calculated using equation 4. The PEG/PEG concentrations in the permeate and retentate are analyzed by a gel permeation chromatography (GPC) system (Shimadzu) using a RID-20A refractive index detector. Based on the diameter of PEG/PEO and their rejection values, the mean effective pore size (μₚ), pore size distribution and MWCO are determined by ignoring interactions between solutes and membrane pores. The mean effective pore size (μₚ) and MWCO of the membrane is determined at the solute rejection of 50% and 90%, respectively. The pore size distribution of the membrane is conducted using the following probability density function based on Equation 3.

$$\frac{d R ( {d}_{p} )}{d {d}_{p}} = \frac{1}{{d}_{p} ln {σ}_{p} \sqrt{2 π}} exp [ - \frac{{( ln {d}_{p} - ln {μ}_{p} )}^{2}}{2 {( ln {σ}_{p} )}^{2}} ] \qquad Equation 3$$

where σₚ is the geometric standard deviation defined as the ratio of pore diameter at 84.13% rejection over that at 50% rejection.

Accordingly, the present disclosure provides highly ordered 2D COF materials with tunable pores and demonstrated the synthesis of multiple pore functionalities. According to embodiments, a cation selective membrane with precise size-selectivity is provided. The synthetic flexibility of this system allows for rational design and synthesis of membrane materials for many different types of separations based on size, charge, hydrophobicity and hydrophilicity among others with potential applications in desalinization, non-protein fouling membranes, fuel cell membranes, redox flow battery membranes, dialysis membranes, gas separation membranes and other technologies requiring membrane separations, with some of them already being pursued in our laboratories.

The above experiments show similar permeance and selectivity for dye molecules of a variety of sizes and charges from aqueous solutions, as well as dyes from organic solutions such as tetrahydrofuran and toluene as a function of size or shape.

Embodiments of the invention provide an entirely new class of ordered two-dimensional (2D) Ordered Functional Nanoporous Materials (OFNMs) with a unique combination of electronic conductivity, gas transport ability, and ion transport properties. In the inventive new materials, the pores having dimensions of from 1.2 nm to 82 nm of longest linear extent across the pore and are synonymously referred to herein as nanopores. The content of PCT/US17/20000 is hereby incorporated by reference.

A novel syntheses method is provided that produces ordered 2D and 3D OFNMs containing chemically modifiable and controllable sized nanopores with many functional groups including charged carboxylates, sulfonates, and protonated amines that will be selective for binding and transporting either cations or anions of any desired size. Specific binding sites for binding catalytic transition or rare earth metals may also be incorporated into the materials for binding and electrocatalysis of specific chemical substrates.

The entirely new configurations and properties associated with the inventive OFNMs formed in embodiments of the invention have a myriad of applications that illustratively include size selective ion transporting membranes for fuel cells, redox flow batteries, electrolyzers, filtration, and desalinization systems. The inventive OFNMs are ideal for battery electrodes due to their rigidity, stability, and electronic conductivity, which have almost no dimensional changes upon charge/discharge cycles, and can be designed with nanopores to be selective for transporting and storing a particular high energy redox species such as Li, Na, Al or Ca. Since the inventive OFNMs are prepared to selectively bind and transport anions, and are also useful as membranes for conventional transition metal containing redox flow batteries. Stacking layers of anion and cation specific materials also enables use of the inventive OFNMs in bipolar membranes for many different applications. The ability to also incorporate electrocatalytic metals into specifically designed binding sites within the same material is a major advancement in fuel cell and electrolyzer designs by incorporating electronic conduction, ion

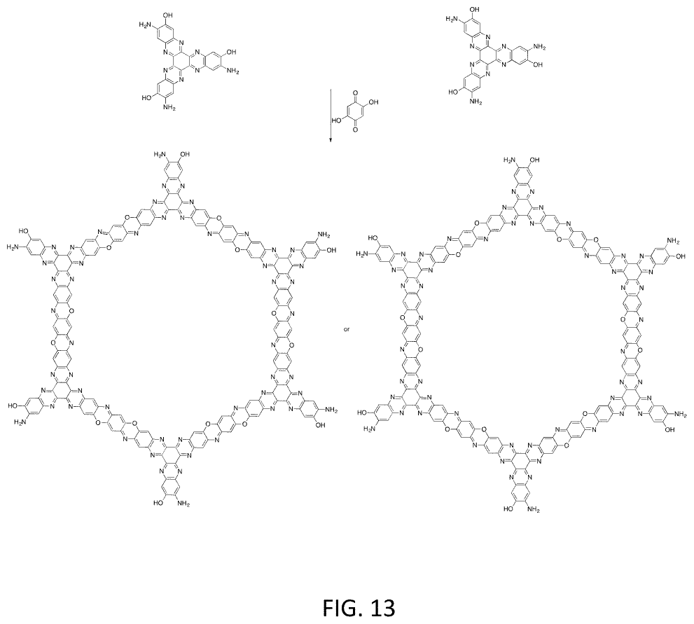

Referring now to the figures, FIG. 14 illustrates a prior art synthesis of reagent a 6,7-diaminoquinoxaline (DAQX) with conditions as shown therein.

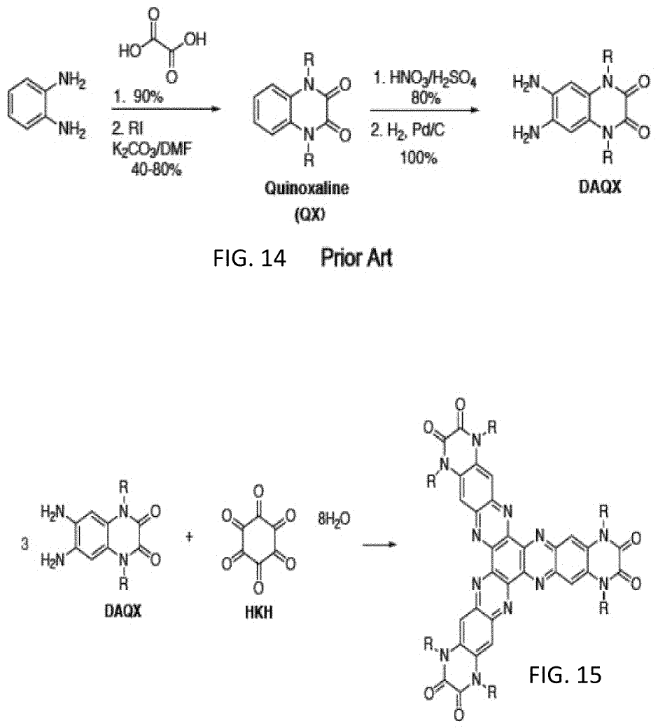

FIG. 15 illustrates an inventive synthesis reaction of DAQX with hexaketocyclohexane (HKH) octahydrate to form a triquinoxaline precursor for an inventive OFNM under standard conventional reaction conditions such as heat, a drying agent to remove water formed in the reaction such as molecular sieves or Dean-Stark distillation. The reaction occurring in a solvent compatible with the reagents that is conventional to the art. R is independently in each occurrence H, Cl, Br, I, C₄H₄S (thiophenyl), SO₃⁻, CO₂⁻, C≡CH, CH═CH₂, NH₂, OH, C≡N, C₁-C₄ alkyl, (CH₂)ₓCH═CH₂, or (CH₂)_{y}CH═CH(CH₂)_{z} where x or (y+z) is an integer of 0 to 4 inclusive, (CH₂)₃CH≡CH, or (CH₂)ₖCH≡C(CH₂)ᵣ where j or (k+r) is an integer of 0 to 4 inclusive.

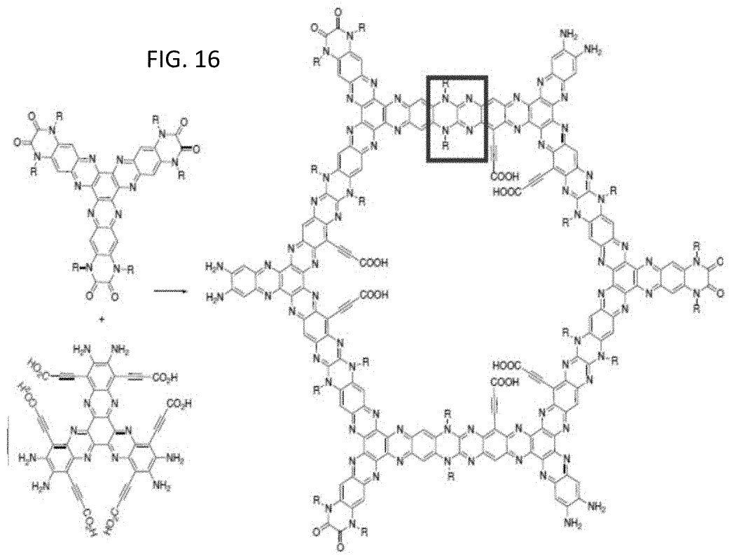

FIG. 16 illustrates an inventive synthesis of a novel OFNM from a triquinoxaline and a hexaazatriphenylenehexacarbonitrile (HAT-hexacarbonitrile). The box highlights in the central uppermost portion of the inventive OFNM encompasses the N—C—N linkage in the pore sidewall. This linkage is particularly robust under a variety of operational conditions including elevated temperatures and extreme redox conditions. The R groups extending both inward and outward relative the pore are amenable to include a variety of functional groups to modify OFNM properties such as the physical pore size, hydrophobicity, pore selectivity, solubility, and a bonding group to a surrounding matrix material.

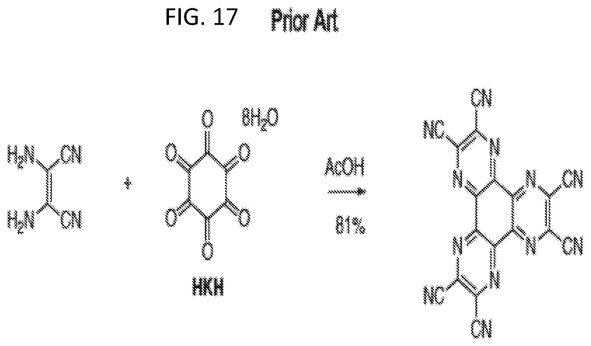

FIG. 17 illustrates a prior art reaction to form (HAT-hexacarbonitrile) per Kanakarajan and Czarnik J. Org. Chem. 1986, 51, 5241.

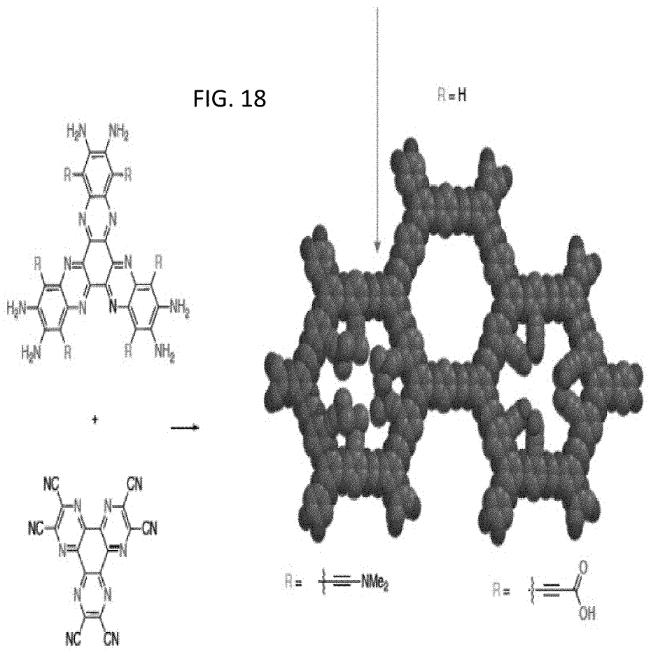

FIG. 18 illustrates the reaction scheme of FIG. 3 and highlighting the variable moieties, R projecting into the pore volume and external to the pore. The arrow shown in FIG. 5 highlights a novel situs of chemical connectivity relative to earlier OFNMs on the exterior of the pore. Specific exemplary R groups are depicted.

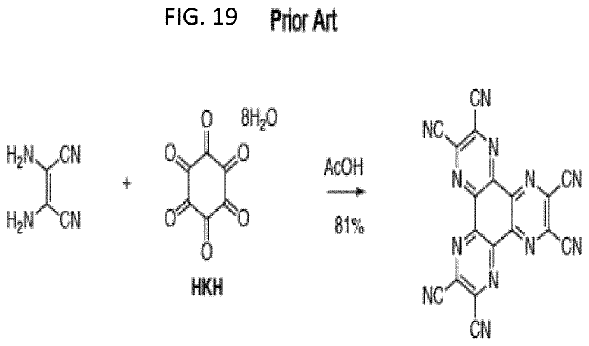

FIG. 19 illustrates an inventive synthesis of a novel 2 pore type OFNM based on a Heck coupling of a triquinoxaline precursor as reagent for a novel OFNM synthesis with functionalized pores, in which R¹ is independently in each occurrence any one of the groups detailed above with respect to R.

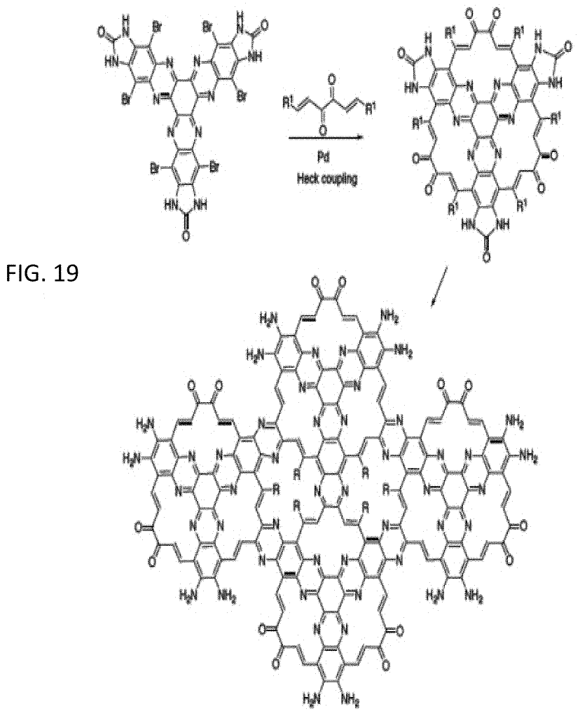

FIG. 20 illustrates the reaction scheme a synthesis of a functionalized OFNM through a reaction scheme involving a functionalized tetraminobenzene and a triquinoxaline precursor. The scheme including heat and deprotonation conditions. An exemplary R² is depicted in FIG. 20. Generally, R² is independently in each occurrence any one of the groups detailed above with respect to R.

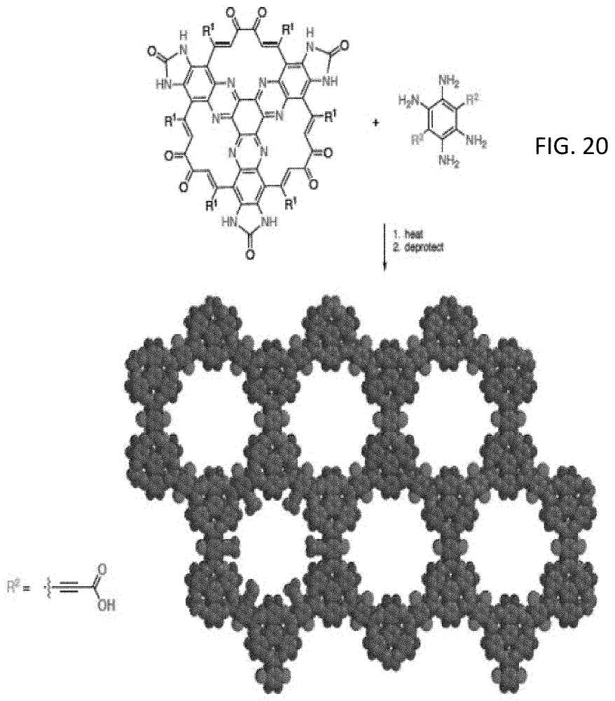

FIG. 21 illustrates a reaction scheme for the self-assembly of a novel dual pore OFNM with reaction conditions provided therein.

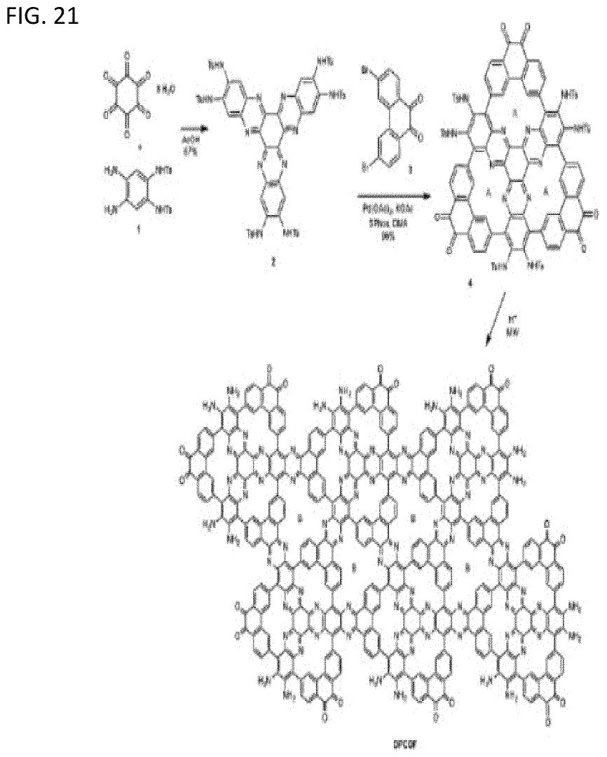

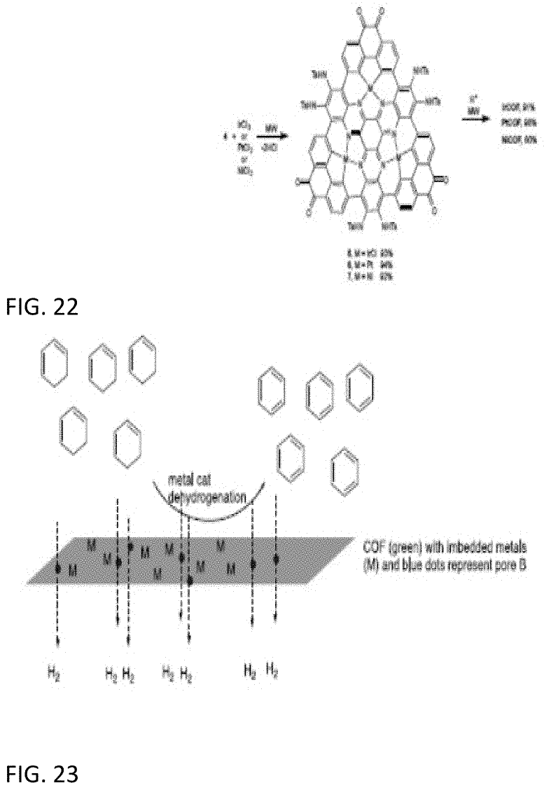

FIG. 22 illustrates a reaction scheme for incorporation of metal ions into monomer 4 depicted in FIG. 23 and as a result, into the dual pore OFNM of FIG. 21.

OFNMs are amenable to use in degasification, a critical step in sensing applications where gas must be extracted to allow for sensing,(1) which include purification of aquaculture water(2) and in petrochemical production and thermal power generation.(3) Degasification is an ideal test for these membranes given that the microporous space in pore B is large enough for the passage of gas molecules but not adequate for permeance of larger solvent molecules. Composite membranes were fabricated by low vacuum assisted filtration of an exfoliated OFNM solution on a 20-nm pore size anodic aluminum oxide membrane support. Liquid filtration tests were performed under a trans-membrane pressure of 10 bar using water and hexane as feeds and pure gases of H₂, O₂, and N₂ under a transmembrane pressure of 5 psi all at room temperature. Table 1 illustrates the results in which clear separation capabilities are obtained. As seen, permeance of both polar and nonpolar solvents are negligible compared to high performance membranes (4,5). Alternatively, permeance of gases thru the same membrane displayed extraordinary GPUs that easily surpass current benchmarks (6-8).

TABLE 1
Solvent and gas separation studies using composite
DPCOF membranes.

| | Liq | | Gas | Gas |
|---|---|---|---|---|
| Liquid | Permeance | Gas | Permeance | Permeance |
| Feed | (L m⁻² h⁻¹ bar¹) | Feed | (GPU) | (L m⁻² h⁻¹ bar¹) |
| Water | 0.91 | H₂ | 91,000 | 245,656 |
| Ethanol | 1.4 | O₂ | 59,000 | 159,271 |
| Hexane | 0.14 | N₂ | 46,000 | 124,177 |

FIG. 23 is schematic showing usage of an OFNM in dehydrogenation and separation of hydrogen gas. A platinum modified OFNM, referred to synonymously as a COF is used to dehydrogenate 1,3-cyclohexadiene as an exemplary feedstock to form H₂ and benzene. The platinum modified OFNM has Pt nanoparticles, organo-platinum compounds, or platinum metal ions imbedded in the COF. Here the OFNM (COF) acts as the support for the platinum and as a membrane to separate the H₂ from the resulting alkene product.

The present invention is further detailed with respect to the following drawings. These figures are not intended to limit the scope of the present invention but rather illustrate certain attributes thereof.

1. An ordered functional nanoporous material (OFMN) composition comprising a pore defined by a sidewall, the sidewall comprising N—C—N linkages therein.

2. The OFNM of claim 1 further comprising R groups extending from the N—C—N linkages, where R is independently in each occurrence H, Cl, Br, I, C₄H₄S (thiophenyl), SO₃⁻, CO₂⁻, C≡CH, CH═CH₂, NH₂, OH, C≡N, C₁-C₄ alkyl, (CH₂)ₓCH═CH₂, or (CH₂)_{y}CH═CH(CH₂)_{z} where x or (y+z) is an integer of 0 to 4 inclusive, (CH₂)₃CH≡CH, or (CH₂)ₖCH≡C(CH₂)ᵣ where j or (k+r) is an integer of 0 to 4 inclusive.

3. A process for synthesis of a reagent of formula: 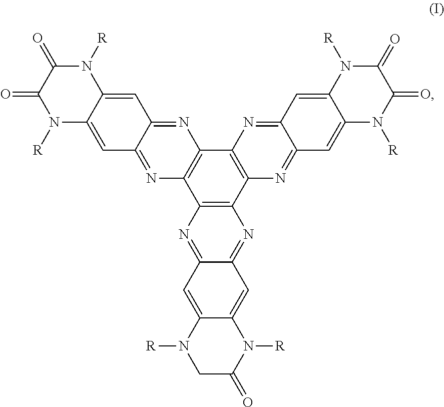
comprising the reaction of a 6,7-diaminoquinoxaline having R groups with hexaketocyclohexane (HKH) octahydrate, where R is independently in each occurrence H, Cl, Br, I, C₄H₄S (thiophenyl), SO₃⁻, CO₂⁻, C≡CH, CH═CH₂, NH₂, OH, C≡N, C₁-C₄ alkyl, (CH₂)ₓCH═CH₂, or (CH₂)_{y}CH═CH(CH₂)_{z} where x or (y+z) is an integer of 0 to 4 inclusive, (CH₂)ⱼCH≡CH, or (CH₂)ₖCH≡C(CH₂)ᵣ where j or (k+r) is an integer of 0 to 4 inclusive.

4. The process of claim 3 further comprising reaction of (I) via a Heck coupling with a reagent having terminal groups R¹, where R¹ is independently in each occurrence H, Cl, Br, I, C₄H₄S (thiophenyl), SO₃⁻, CO₂⁻, C≡CH, CH═CH₂, NH₂, OH, C≡N, C₁-C₄ alkyl, (CH₂)ₓCH═CH₂, or (CH₂)_{y}CH═CH(CH₂)_{z} where x or (y+z) is an integer of 0 to 4 inclusive, (CH₂)₃CH≡CH, or (CH₂)ₖCH≡C(CH₂)ᵣ where j or (k+r) is an integer of 0 to 4 inclusive to yield a molecule of the formula:

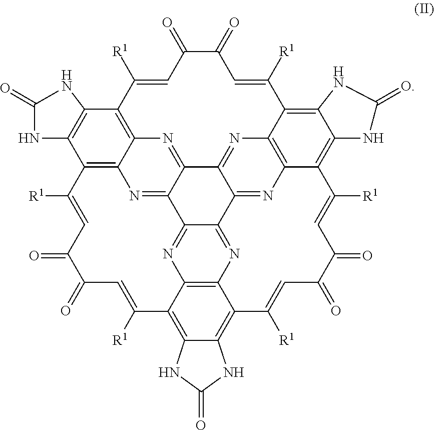

5. The process of claim 4 further comprising reaction (II) with a tetraminobenzene.

6. The OFNM of claim 1 having at least two pores

7. The OFNM of claim 6 wherein the two pore OFNM is formed by a process of self assembly.

8. The OFNM of claim 7 further comprising incorporating a metal therein.

9. A process of degasification comprising:
extracting a gas from a mixture by exposing the mixture to an OFNM to selectively pass the gas therethrough.

10. A process of dehydrogenation comprising:
exposing an aliphatically unsaturated feedstock to platinum modified OFNM under conditions to form hydrogen; and
selectively passing the hydrogen through the platinum modified OFNM.

11. The process of claim 10 wherein platinum in the platinum modified OFNM is in the form of Pt nanoparticles.

12. The process of claim 10 wherein platinum in the platinum modified OFNM is in the form of organo-platinum compounds.

13. The process of claim 10 wherein platinum in the platinum modified OFNM is in the form of platinum metal ions.
